(19) United States
(12) Patent Application Publication
(10) Pub. No.: US 2026/0341466 A1
(43) Pub. Date: Oct. 29, 2026

(54) MACHINE LEARNING FOR NUTRITION PLAN GENERATION

(71) Applicant: MatrixCare, Inc., Bloomington, MN (US)

(72) Inventors: Amy WOOTTON, Loxahatchee, FL (US); Zheng Yuan ZHU, Toronto (CA); Bhavesh Bhagvat TRIVEDI, Acworth, GA (US)

(21) Appl. No.: 19/634,321

(22) Filed: Mar. 31, 2026

Related U.S. Application Data
(60) Provisional application No. 63/793,568, filed on Apr. 23, 2025.

(51) Int. Cl. G16H 20/60; G06Q 50/12

(52) U.S. Cl. CPC G16H 20/60; G06Q 50/12

(57) ABSTRACT

Techniques for improved machine learning are provided. A plurality of menu definitions are accessed, where each respective menu definition of the plurality of menu definitions specifies one or more meals. A request to generate a new menu definition is received, where the request specifies a nutritional constraint for the new menu definition. Using a machine learning model, the new menu definition is generated based on the plurality of menu definitions and the nutritional constraint. The new menu definition is output via one or more graphical user interfaces, and implementation of the new menu definition is facilitated.

### CROSS-REFERENCE TO RELATED APPLICATIONS

This application claims benefit of co-pending United States provisional patent application Serial No. 63/793,568 filed Apr. 23, 2025. The aforementioned related patent application is herein incorporated by reference in its entirety.

### TECHNICAL FIELD

The present disclosure relates generally to machine learning, and more particularly, to use of machine learning to generate nutrition plans.

In a wide variety of care settings, users receive varying amounts and types of support to ensure that their nutritional needs are met. For example, the support may include meal planning, grocery shopping, meal preparation, meal cleanup, and the like. Often, the degree of the support can vary. Further, the context of the users receiving such support may vary. For example, nutritional support may be provided in residential care settings (e.g., for residents residing in a care facility, such as a long-term residential facility), home-healthcare settings (e.g., where meals and/or ingredients can be delivered to users), and the like. In many cases, users may have disparate dietary or nutritional needs or preferences. The vast number and assortment of combinations and needs renders computational optimization effectively impossible, even using modern computing resources. Accordingly, proper nutrition planning today relies on extensive manual effort.

Improved systems and techniques to use machine learning for nutrition planning are desired.

### SUMMARY

According to some implementations of the present disclosure, a method includes: accessing a plurality of menu definitions, wherein each respective menu definition of the plurality of menu definitions specifies one or more meals; receiving a request to generate a new menu definition, wherein the request specifies a nutritional constraint for the new menu definition; generating, using a machine learning model, the new menu definition based on the plurality of menu definitions and the nutritional constraint; outputting the new menu definition via one or more graphical user interfaces; and facilitating implementation of the new menu definition.

According to some implementations of the present disclosure, a system includes a control system and a memory. The control system includes one or more processors. The memory has stored thereon machine readable instructions. The control system is coupled to the memory, and any one of the methods disclosed herein is implemented when the machine executable instructions in the memory are executed by at least one of the one or more processors of the control system.

Other aspects provide processing systems configured to perform the aforementioned method as well as those described herein; non-transitory, computer-readable media comprising instructions that, when executed by one or more processors of a processing system, cause the processing system to perform the aforementioned methods as well as those described herein; a computer program product embodied on a computer-readable storage medium comprising code for performing the aforementioned methods as well as those further described herein; and a processing system comprising means for performing the aforementioned methods as well as those further described herein.

The above summary is not intended to represent each implementation or every aspect of the present disclosure. Additional features and benefits of the present disclosure are apparent from the detailed description and figures set forth below.

### BRIEF DESCRIPTION OF THE DRAWINGS

So that the manner in which the above-recited features of the present disclosure can be understood in detail, a more particular description, briefly summarized above, may be had by reference to aspects, some of which are illustrated in the appended drawings. It is to be noted, however, that the appended drawings illustrate only certain typical aspects of this disclosure and are therefore not to be considered limiting of its scope, for the description may admit to other equally effective embodiments.

FIG. 1 depicts an example workflow for using machine learning to generate nutrition plans, according to some embodiments of the present disclosure.

FIG. 2 depicts an example workflow for training machine learning models to generate nutrition plans, according to some embodiments of the present disclosure.

FIG. 3 depicts an example architecture for nutrition planning, according to some embodiments of the present disclosure.

FIG. 4 is a flow diagram depicting an example method for using machine learning to generate nutrition plans, according to some embodiments of the present disclosure.

FIG. 5 is a flow diagram depicting an example method for training machine learning models to generate nutrition plans, according to some embodiments of the present disclosure.

FIG. 6 is a flow diagram depicting an example method for nutrition planning, according to some embodiments of the present disclosure.

FIG. 7 depicts an example computing device configured to perform various aspects of the present disclosure, according to some embodiments disclosed herein.

While the present disclosure is susceptible to various modifications and alternative forms, specific implementations and embodiments thereof have been shown by way of example in the drawings and will herein be described in detail. It should be understood, however, that it is not intended to limit the present disclosure to the particular forms disclosed, but on the contrary, the present disclosure is to cover all modifications, equivalents, and alternatives falling within the spirit and scope of the present disclosure as defined by the appended claims.

### DETAILED DESCRIPTION

Embodiments of the present disclosure generally provide techniques for using machine learning for nutrition planning.

In some embodiments, machine learning models can be generated and/or trained to create nutrition plans in the form of menu definitions, where a menu definition specifies a set of meals (e.g., breakfast, lunch, snack, and/or dinner) for a set of days (e.g., one week, one month, and the like) and each respective meal includes a respective set of meal components (e.g., a protein component, a starch component, a vegetable component, a fruit component, an entrée component, and the like). In some embodiments, the system can further generate the nutrition plan based on defined dietary and/or nutritional constraints.

In some embodiments, the nutritional constraint corresponds to or comprises a therapeutic dietary restriction and/or consistency restriction. That is, the nutritional constraint may indicate various restrictions for the meal plan based on the diet needs of the user(s) (e.g., specifying a maximum amount of sugar per day, a minimum amount of protein, requiring inclusion and/or exclusion of particular ingredients such as gluten, and the like). In some embodiments, the constraints may be expressed in the form of therapeutic diet plans, such as a meal plan for users having diabetes (e.g., a “diabetes diet”), a meal plan for users with kidney disease (e.g., a “renal diet”), a meal plan for users with heart concerns (e.g., a “cardiac diet”), a meal plan for users without any of the specified concerns (e.g., a “default diet”), and the like. In some embodiments, the constraints may be expressed in the form of consistency (e.g., to ensure consistency in the generated meals).

In some embodiments, a machine learning system may be prompted to use machine learning models to generate a menu definition based on a request specifying context such as the number of users for whom the definition is being created (e.g., indicating that a menu to feed five hundred users is desired), the duration of the definition (e.g., indicating that the menu should cover one week and/or should include a defined number of meals), the nutritional constraint(s) of the user(s), and the like. In some embodiments, the request may also specify other constraints or preferences, such as a target or preferred expense (e.g., per meal, per day, per user, and/or a total cost of the entire plan), a target or preferred food item rotation, and the like.

The machine learning system may use models trained based on historic menu definitions (e.g., meal plans created manually) to generate the requested new menu definitions. In some embodiments, the requesting user may provide feedback in the form of modifications to the newly generated menu definitions. For example, the user may replace one meal with another, modify one or more meal component(s) of one or more meal(s), and the like. In some embodiments, based on such modifications, the machine learning system may refine or modify the machine learning model(s) (e.g., to increase the probability that the modified menu will be generated in the future and/or reduce the probability that the originally generated menu will be generated again in the future).

In some embodiments, after generating a new menu, the machine learning system (or another system) may facilitate implementation of the menu. For example, in some embodiments, the machine learning system may order or request one or more food items (e.g., ingredients, meal components, and/or pre-prepared meals) to satisfy the menu definition. That is, the machine learning system may order and/or schedule delivery of one or more food items used to prepare one or more meal(s) included in the definition, allowing the requesting user to prepare the meal(s) in accordance with the menu. In some embodiments, implementing the menu may include scheduling delivery of the relevant food item(s) at the relevant time and/or location (e.g., such that the ingredients used for the generated set of meals for a given day arrive at the indicated location in time for preparation of the set of meals, such as arriving the day before).

In some embodiments, using machine learning models to generate the menu definitions can enable the computing systems to dynamically generate meal plans with reduced computational expense, as compared to conventional rules-based approaches. For example, due to the variety of nutritional constraints that may be applicable, an overwhelming number of rules may be required to implement a conventional meal planning system. Such a vast number of rules may impractical to implement using available computing resources (e.g., as storing these rules would rely on large storage and/or memory volumes, and evaluating these rules would incur substantial latency and computational expense, as well as power dissipation, heat generation, and the like).

Further, given the large number of components that can be included in a given menu definition, the number of possible combinations is effectively infinite. For example, in addition to the vast number of alternatives that exist for any given meal component (e.g., the number of vegetable alternatives), the menu definition may further specify the means or style of preparation for such components (e.g., whether the vegetable should be sautéed, boiled, braised, grilled, roasted, steamed, stir-fried, and so on). The number of possible combinations for even a small number of meals (e.g., a single day) causes the meal planning process to rapidly becomes intractable (e.g., relying on evaluation of millions or billions of combinations for even small nutrition plans).

However, by training machine learning models using previously prepared meal definitions, the models can efficiently learn to dynamically generate new plan definitions without explicit generation and/or evaluation of the vast number of alternatives that are considered by conventional systems. That is, using such machine learning models as discussed in more detail below renders the problem computationally tractable, such that readily available computing systems are able to effectively generate nutrition plans with reduced computational expense, as compared to prior solutions. This improved generation process consumes fewer computational resources, incurs reduced latency, and generally enables existing systems to generate useful output that these systems were simply unable to generate using conventional approaches.

### Example Workflow for Using Machine Learning to Generate Nutrition Plans

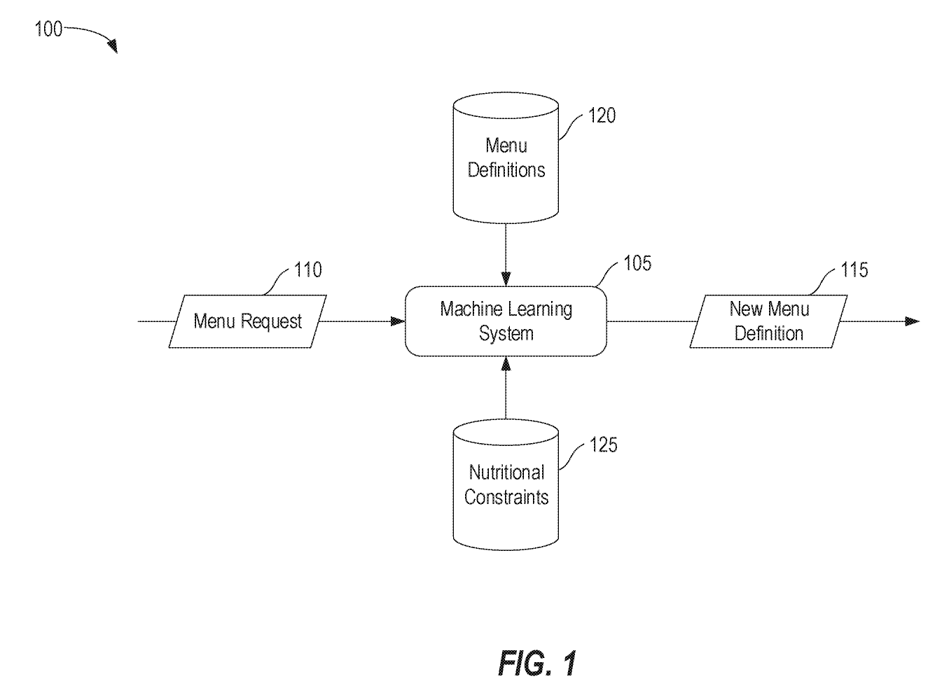

FIG. 1 depicts an example workflow 100 for using machine learning to generate nutrition plans, according to some embodiments of the present disclosure.

In the illustrated example, a machine learning system 105 accesses a menu request 110 and uses one or more machine learning models to generate a new menu definition 115. As used herein, “accessing” data may generally include receiving, requesting, retrieving, obtaining, collecting, generating, or otherwise gaining access to the data. For example, the machine learning system 105 may receive the menu request 110 from a requesting entity, which may correspond to a user (e.g., a caregiver or other individual tasked with performing or facilitating meal planning), a computational entity (e.g., a software application), and the like. In some embodiments, the menu request 110 is accessed via an application programming interface (API). For example, the machine learning system 105 may be implemented as a remote service (e.g., a cloud-based application), and users may interact with the machine learning system 105 (e.g., to generate new menu definitions 115) via one or more APIs.

Although not depicted in the illustrated example, in some embodiments, user permissions or approvals may be evaluated (e.g., by the machine learning system 105) prior to generating a new menu definition 115 based on the menu request. For example, the machine learning system 105 may evaluate permission of the user that submitted the menu request 110 to ensure that only users with sufficient authority can access the machine learning system 105 to generate and/or modify menus.

Although the machine learning system 105 is depicted as a discrete system for conceptual clarity, in some embodiments, the operations of the machine learning system 105 may be combined or distributed across any number and variety of systems, and may be implemented using hardware, software, or a combination of hardware and software.

In some embodiments, the menu request 110 can be used to specify the context of the desired new menu definition 115, such as the number of meals to include (e.g., the number of meals per day and/or the number of days), the number of users who will receive the meals, any nutritional constraints for the new menu definition 115, preferences or targets for one or more components of the new menu definition 115 (e.g., a target price per meal, per day, and/or per user), and the like. In some embodiments, the menu request 110 may specify constraints relating to the contents of the menu definition, such as that the new menu should include at least one specific meal (e.g., specifying a meal that is popular among the residents of a given facility), that the menu should not include at least one meal (e.g., because the meal is not popular in the facility, or is culturally inappropriate for the target facility), and the like.

In some embodiments, the menu request 110 comprises unstructured natural language text, such as “generate a one-week diabetic meal plan for three hundred users.” In some embodiments, the menu request 110 may additionally include structured information, such as specifying the particular nutritional constraint(s) from a set of available constraints.

The new menu definition 115 generally comprises, for each respective day of a set of one or more days, a set of one or more meals. In some embodiments, as discussed above, each respective meal of the set of one or more meals specifies a set of meal components (e.g., specific selections for each component group). The component groups may generally be defined by nutritional role (e.g., a “protein” component, a “starch” component, a “vegetable” component, and the like) and/or by the role the component plays in the meal (e.g., “entrée,” “side,” “desert,” “appetizer,” and the like). In some embodiments, each meal component specifies the particular dish (e.g., “potatoes au gratin”) and/or ingredients therein (e.g., “potatoes, milk, cheese, cream, butter”). In some embodiments, each meal component may specify the method of preparation (e.g., “grilled”) in addition to listing the food item itself (e.g., “asparagus”). In some embodiments, the meal components may specify portions for each item or ingredient (e.g., “one teaspoon of salt”).

In the illustrated example, the machine learning system 105 may also access menu definitions 120. The menu definitions 120 generally correspond to example menus (e.g., where each menu definition 120 comprises a set of meals, each comprising one or more ingredients or components). For example, some or all of the menu definitions 120 may be previously generated menus (e.g., by the machine learning system 105 or using other techniques). In some embodiments, the menu definitions 120 include one or more manually created and/or curated menus (e.g., designed by nutritionists).

In some embodiments, the machine learning model(s) may be trained and/or refined based on the menu definitions 120. That is, the machine learning system 105 may receive the menu definitions 120 to train or refine the models, and the generation of the new menu definition 115 may be performed using the trained models. In some embodiments, the machine learning system 105 does not evaluate or process the menu definitions during runtime (e.g., after training).

In some embodiments, the machine learning system 105 generates the new menu definition 115 during runtime based at least in part on the menu definitions 120. For example, the menu definitions 120 may define a set of meal(s) and/or meal components, and the machine learning system 105 may generate the new menu definition 115 based on these meals. As one example, the machine learning system 105 may generate the menu for a given day by selecting or copying a one-day menu from the set of menu definitions 120. As another example, the machine learning system 105 may generate the menu for a given meal by selecting or copying a meal included in the menu definitions 120. As yet another example, the machine learning system 105 may generate the menu for a given meal by selecting individual meal components from any number of different meals in the menu definitions 120. Stated differently, the machine learning system 105 may generate the new menu definition 115 based in part on dynamic selection of the various components of the menu from an already defined set of alternatives included in the menu definitions 120.

For example, suppose the menu definitions 120 include a meal “Meal A” having components “Starch A,” “Vegetable A,” and “Protein A” as well as a “Meal B” having components “Starch B,” “Vegetable B,” and “Protein B.” In some embodiments, the machine learning system 105 may generate the new menu definition 115 by selecting “Meal A” and/or “Meal B” for one or more days, and/or by selecting components therefrom (e.g., generating a new “Meal C” having components “Starch A,” “Vegetable B,” and “Protein A”).

In the illustrated example, the machine learning system 105 also accesses a set of defined nutritional constraints 125. In some embodiments, the nutritional constraints may specify the rules or restrictions applicable to various types of therapeutic diets. For example, the nutritional constraints 125 may specify the limitations or preferences for a “cardiac diet” as one that includes low saturated fats, cholesterol, sodium, and/or sugar (e.g., each below one or more daily thresholds), while including fruits, vegetables, and lean protein (e.g., each meeting one or more daily thresholds).

In some embodiments, in a similar manner to the menu definitions 120, the machine learning system 105 may analyze the nutritional constraints 125 during a training phase, but may not use them during runtime. In other embodiments, the machine learning system 105 may evaluate the nutritional constraints 125 during runtime (e.g., to look up the definition of any constraints indicated in the menu request 110, and/or to confirm that a generated new menu definition 115 satisfies the intended constraints after generation).

In some embodiments, the machine learning system 105 uses one or more trained machine learning models to generate the new menu definition 115. The particular architecture of the machine learning model(s) may vary depending on the particular implementation. In some embodiments, as discussed in more detail below, the machine learning model(s) are trained based on historical menu definitions.

For example, in some embodiments, the machine learning system 105 may use architectures such as a random forest to generate or select each component of the new menu definition 115. In some embodiments, the machine learning system 105 may use a natural language model, such as a large language model (LLM) to generate the new menu definition 115 based on natural language menu requests 110.

In the illustrated example, the machine learning system 105 outputs the new menu definition 115 (e.g., to a user such as nutritionist) for review and/or approval. Although not depicted in the illustrated example, in some embodiments, the machine learning system 105 may receive modifications or other feedback relating to the new menu definition 115, and may use this feedback to refine the model(s). In some embodiments, the machine learning system 105 may facilitate implementation of the new menu definition 115 (immediately or after approval by the user).

As one example, the machine learning system 105 may schedule delivery of the indicated food items at appropriate times based on the new menu definition 115. For example, the machine learning system 105 may identify the food items (e.g., pre-prepared food items, raw ingredients, and the like) that will be used on a given day (based on the new menu definition 115 for that day), and may schedule delivery of these items prior to the given day (e.g., on the day before).

In this way, user(s) need not manually create menus, track nutritional constraints, or manage item ordering and delivery.

### Example Workflow for Training Machine Learning Models to Generate Nutrition Plans

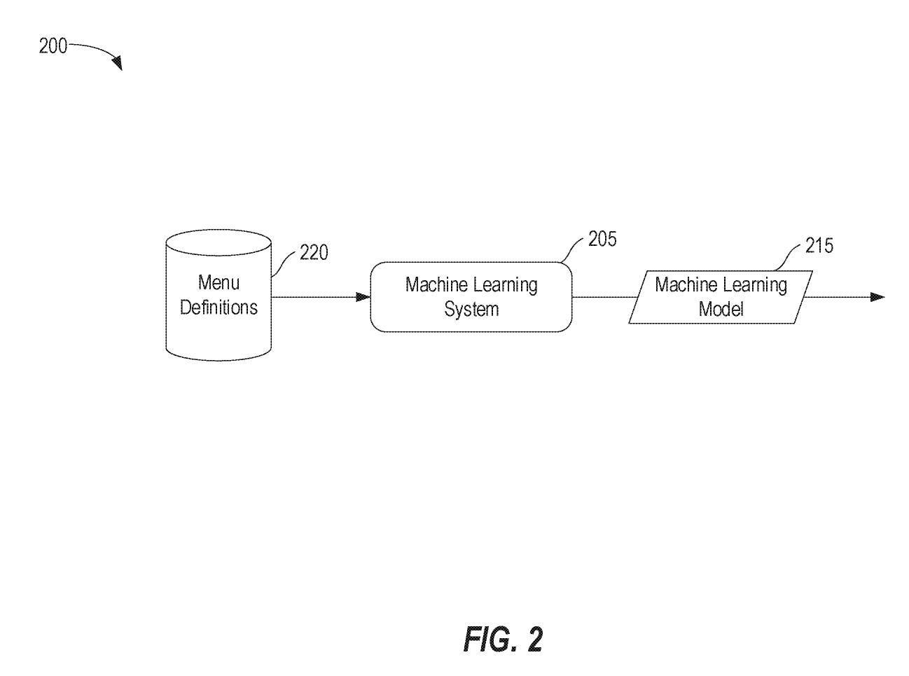

FIG. 2 depicts an example workflow 200 for training machine learning models to generate nutrition plans, according to some embodiments of the present disclosure.

In the illustrated example, a machine learning system 205 accesses a set of menu definitions 220 to train one or more machine learning models 215 to generate a new menu definition. The machine learning system 205 may correspond to the machine learning system 105 of FIG. 1 (that is, a single system may perform by training and runtime inferencing), or may be a separate training system (that is, one system may train the models while another uses the trained models during runtime).

Although the machine learning system 205 is depicted as a discrete system for conceptual clarity, in some embodiments, the operations of the machine learning system 205 may be combined or distributed across any number and variety of systems, and may be implemented using hardware, software, or a combination of hardware and software.

The machine learning system 205 may access the menu definitions 220 from a variety of sources. Although a single repository of menu definitions 220 is depicted for conceptual clarity, in some embodiments, the machine learning system 205 may access the menu definitions 220 from multiple repositories,

In some embodiments, the menu definitions 220 (which may correspond to the menu definitions 120 of FIG. 1) generally correspond to example menus (e.g., where each menu definition 220 comprises a set of meals, each comprising one or more ingredients or components). For example, some or all of the menu definitions 220 may be previously generated menus (e.g., by the machine learning system 205 or using other techniques). In some embodiments, the menu definitions 220 include one or more manually created and/or curated menus (e.g., designed by registered dieticians, nutritionists, and the like).

The machine learning system 205 generally trains the machine learning model 215 using the set of menu definitions 220 as exemplars. The particular operations used to train the machine learning model 215 may vary depending on the particular implementation and architecture used. For example, if the machine learning model 215 uses a language model architecture (e.g., an LLM), the machine learning system 205 may use the menu definitions 220 (or natural language strings generated based on the menu definitions 220, such as listing the meals and components thereof in sequence) during the fine-tuning phase to train the machine learning model 215 such that the machine learning model 215 learns patterns and relationships (e.g., relationships between meal components within a meal, meals in a day, and/or meals across days) from historical menu definitions 220. In some embodiments, the menu definitions 220 may additionally include rating information (e.g., scores generated based on user feedback indicating their satisfaction with one or more components of one or more of the specified meals, one or more meals in the plan (e.g., a combination of components), and/or with the combination of meals (e.g., in a day or a week). In some embodiments, the machine learning model 215 may further learn patterns in the menu definitions and how they correlate with the indicated satisfaction scores (e.g., to learn to generate menu definitions that will result in higher scores). Based on these relationships, the machine learning model 215 can then generate new menu definitions that are in some ways similar to the manually created menu definitions 220.

As discussed above, the machine learning model 215 may process a variety of data during runtime to generate new menu definitions. For example, if the model is an LLM, the historical menu definitions 220 may be used as the context for a prompt such as “generate a new menu for renal patients based on these example menus.”

### Example Architecture for Nutrition Planning

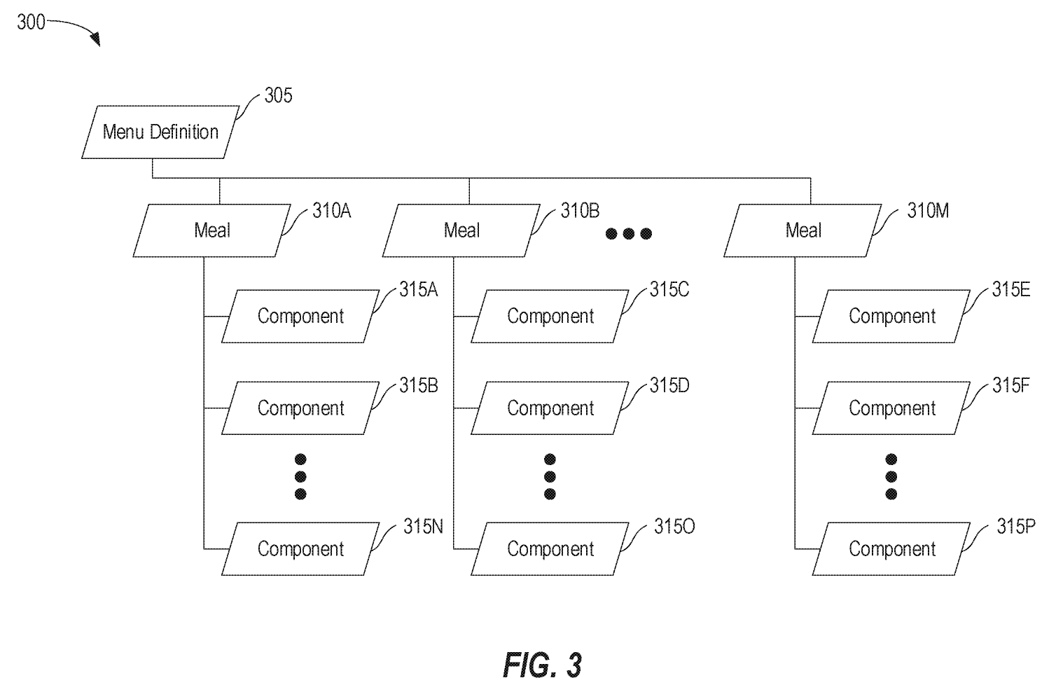

FIG. 3 depicts an example architecture 300 for nutrition planning, according to some embodiments of the present disclosure. In some embodiments, the architecture 300 is used by a machine learning system, such as the machine learning system 105 of FIG. 1 and/or the machine learning system 205 of FIG. 2. In some embodiments, the architecture 300 is used to generate a structured nutrition plan (e.g., menu definition) based on unstructured or disparate components. In other embodiments, the architecture 300 may be used to generate an unstructured string (e.g., a natural language string) based on a structured meal plan.

In the illustrated example, a menu definition 305 (which may correspond to the menu definitions 120 of FIG. 1 and/or 220 of FIG. 2) is a data structure comprising or indicating a set of meals 310A-M. Although the illustrated example depicts three meals 310, there may be any number of meals in a given menu definition 305. In some embodiments, the meals 310 may be labeled or defined based on their intended role. For example, rather than generically specifying meals 310, the menu definition 305 may specify, for each day, a “breakfast” meal, a “lunch” meal, a “dinner” meal, and the like.

Further, in the illustrated example, each meal 310 comprises or indicates a corresponding set of meal components 315A-P. Specifically, the meal 310A includes components 315A, 315B, and 315N, the meal 310B includes components 315C, 315D, and 315O, and the meal 310M includes components 315E, 315F, and 315P. Although each meal 310 is depicted as containing three components 315, in embodiments, each meal 310 may include any number of components 315 (including the more or less than any other meal 310 in the menu definition 305).

In some embodiments, during training, meal definitions formatted according to any suitable format (including unstructured text, such as a string of natural language) may be used to generate structured menu definitions 305 in order to facilitate training of the machine learning model(s). That is, depending on the model architecture used, creating a structured menu definition 305 may improve or enable training that is more robust and accurate than using unstructured text. In other embodiments, structured menu definitions 305 may be preprocessed to generate unstructured input (e.g., a natural language string), depending on the model architecture. For example, for training a language model, the menu definition 305 may be parsed to generate a string such as “The first meal includes Component 315A, Component 315B, and Component 315N. The next meal includes Component 315C, Component 315D, and Component 315O,” and so on.

Similarly, in some embodiments, the architecture 300 may be used during runtime generation of new menu definitions depending on the output of the model and the desired formatting. For example, if the model generates unstructured output (e.g., natural language text), such as if the model is an LLM, the machine learning system may use the architecture 300 to generate a structured representation of the proposed meal plan. This may facilitate improved planning and scheduling (e.g., to readily aggregate the information to determine the total number or quantity of each component that is needed, to determine the timing of each delivery, and the like).

As another example, if the model generates a structured output (e.g., selecting the individual components 315 for each meal 310 and outputting a structured menu definition 305), the machine learning system may use the architecture 300 to generate an unstructured representation (e.g., human-readable natural language text), such as for distribution to the user(s) who will prepare the meals, to the user(s) who will consume the meals, and the like. For example, the machine learning system may generate an output string such as “Dinner on Monday will be Meal 310A, which includes Component 315A, Component 315B, and Component 315N.”

### Example Method for Using Machine Learning to Generate Nutrition Plans

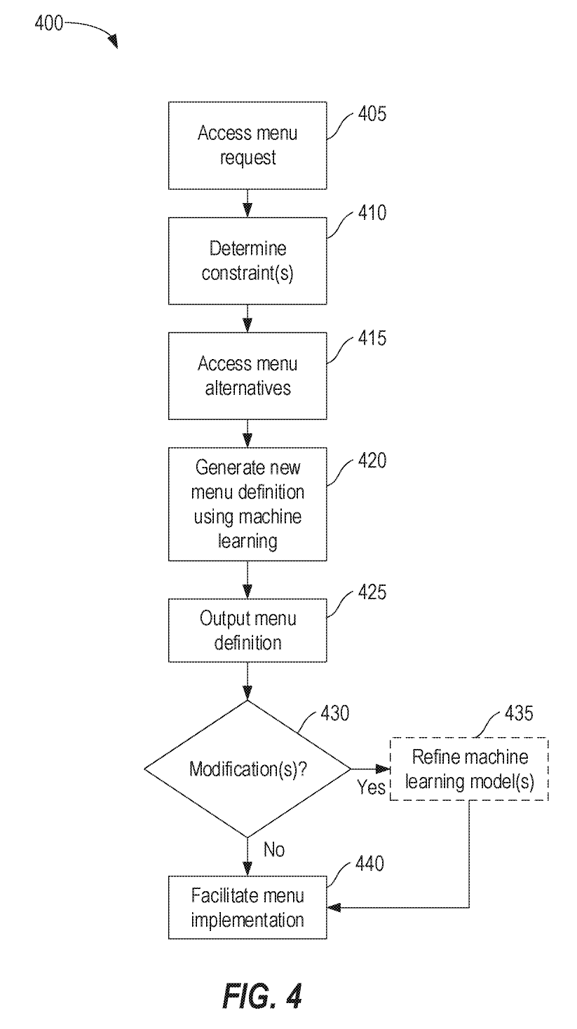

FIG. 4 is a flow diagram depicting an example method 400 for using machine learning to generate nutrition plans, according to some embodiments of the present disclosure. In some embodiments, the method 400 is performed by a machine learning system, such as the machine learning system 105 of FIG. 1 and/or the machine learning system 205 of FIG. 2. In some embodiments, the method 400 provides additional detail for the workflow 100 of FIG. 1.

At block 405, the machine learning system accesses a menu request (e.g., the menu request 110 of FIG. 1). In some embodiments, as discussed above, the machine learning system may receive the menu request from a remote user and/or system (e.g., via an API). The menu request may generally specify the context for the menu, such as the desired number of meals, the duration of the menu, any nutritional constraints for the menu, any preferences relating to the menu (e.g., target costs), and the like. As discussed above, the menu request may include natural language text (e.g., a string describing the context) and/or structured input (e.g., selections for a variety of fields).

At block 410, the machine learning system determines any constraint(s) applicable to the new menu. For example, as discussed above, the request may specify nutritional constraints based on the therapeutic diet(s) of the user(s) who will consume the meals. As one example, the request may specify to generate a one-week long menu for fifteen diabetic users and ten users on a renal diet. In some embodiments, as discussed above, the machine learning system may evaluate a set of defined constraints (e.g., the nutritional constraints 125 of FIG. 1) based on the input to identify the specific rules or preferences that are applicable to the given constraints (e.g., the maximum amount of sodium per day that should be included in the menu).

At block 415, the machine learning system accesses a set of menu alternatives (e.g., the menu definitions 120 of FIG. 1) to assist in generating the new menu. For example, as discussed above, the machine learning system may identify previously generated menus in order to generate a new menu including the components of the previous menus and/or incorporating the structure and relationships indicated in the previous menus. In some embodiments, the particular manner in which the menu alternatives are used may vary depending on the particular implementation and model architecture used. For example, in the case of a language model, the menu alternatives may be used as contextual input to the model (e.g., asking the model to generate a new menu based on the provided set of examples). As another example, the menu alternatives may be used as the specific alternatives that can be used (e.g., where the model is trained to dynamically generate menus by selecting components from the list of alternatives until the full set of meals is created).

At block 420, the machine learning system generates a new menu definition (e.g., the new menu definition 115 of FIG. 1) based on the menu request, constraint(s), and/or menu alternatives. In some embodiments, as discussed above, the constraint(s) may themselves be used as input to the machine learning model. In some embodiments, the constraints may be used to select which model, from a set of trained models, should be used. For example, the machine learning system may train and/or use a respective model for each respective therapeutic diet (e.g., a first model trained to generate diabetic menus, a second for renal menus, and so on).

Although not illustrated in the example method 400, in some embodiments, the machine learning system may use the determined nutrition constraints to perform a secondary rules-based evaluation of the machine learning-generated meal plan. That is, after a plan is generated using the machine learning model(s), the machine learning system may evaluate the plan in view of the determined constraints to ensure that the generated plan aligns with the indicated diet (e.g., that the cholesterol does not exceed a defined daily maximum). If any aspects of the plan fail this evaluation, the machine learning system may make appropriate modifications (e.g., replacing one or more meal components with a component having lower cholesterol) to remedy the deficiencies.

At block 425, the machine learning system outputs the menu definition (e.g., to the individual that requested generation of the menu). For example, the machine learning system may output the menu via a graphical user interface (GUI). In some embodiments, as discussed above, the machine learning system may use the architecture 300 of FIG. 3 to reformat the menu prior to outputting it. For example, if the model generates unstructured output, the machine learning system may generate a structured representation of the menu definition, and output this structure. As another example, if the model generates a structured representation, the machine learning system may generate a corresponding unstructured text string describing the menu, and output this string.

At block 430, the machine learning system determines whether one or more modifications were received for the menu. For example, as discussed above, a user may manually modify or change one or more components of the menu, such as to switch meal(s) between days, to switch components between meals, to replace meal(s) and/or components with new ones, and the like. If no modifications are received (e.g., the user approved the menu without modification, the method 400 continues to block 440. If one or more modifications are received, the method 400 continues to block 435.

At block 435, the machine learning system optionally refines the machine learning model(s) based on the modifications. For example, if the user replaced one meal component with another, the machine learning system may learn that the replaced meal component should not be used in combination with the other components already present in the given meal, and/or that the newly added component should be included in such meals. This can allow the machine learning system to learn based on the preferences of the particular user(s) and/or environments. The method 400 then continues to block 440.

At block 440, the machine learning system facilitates implementation of the menu. As discussed above, facilitating implementation of the menu may include, for example, requesting or ordering food items that will be used to implement the menu (e.g., submitting a purchase order, scheduling delivery of such items, and the like).

### Example Method for Training Machine Learning Models to Generate Nutrition Plans

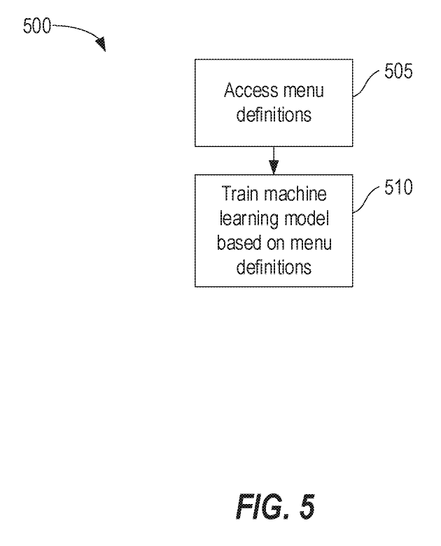

FIG. 5 is a flow diagram depicting an example method 500 for training machine learning models to generate nutrition plans, according to some embodiments of the present disclosure. In some embodiments, the method 500 is performed by a machine learning system, such as the machine learning system 105 of FIG. 1 and/or the machine learning system 205 of FIG. 2. In some embodiments, the method 500 provides additional detail for the workflow 200 of FIG. 2.

At block 505, the machine learning system accesses a set of historical menu definitions (e.g., the menu definitions 220 of FIG. 2). As discussed above, the menu definitions may generally specify the components of one or more previous meals that are part of one or more previous menus (e.g., menus that were previously used for nutrition planning). In some embodiments, as discussed above, the machine learning system may use the architecture 300 of FIG. 3 to reformat the menu definitions, as desired. For example, the machine learning system may convert structured definitions to unstructured text (e.g., for training a language model) and/or may convert unstructured text definitions to a structured menu definition (e.g., for training a neural network model).

At block 510, the machine learning system trains a machine learning model based on the model definitions. As discussed above, the particular operations used to train the model may vary depending on the particular architecture and implementation. For example, if the model is a language model (e.g., an LLM), the machine learning system may use each menu definition as the target or contextual information that the model should seek to generate (e.g., for the task-training during fine tuning). As another example, for other architectures, the machine learning system may use the menu definitions to learn patterns and relationships among the various meals and/or meal components reflected in the historical plans, such as to learn patterns in the variety of vegetables that are served in a given day and/or sequential days.

As discussed above, once the model is trained, it can be deployed for runtime use. Generally, deploying the machine learning model may include any operations used to prepare or provide the model for inferencing, such as transmitting the learned parameter(s) to an inferencing system, instantiating the model locally (e.g., in memory) for use, and the like.

### Example Method for Nutrition Planning

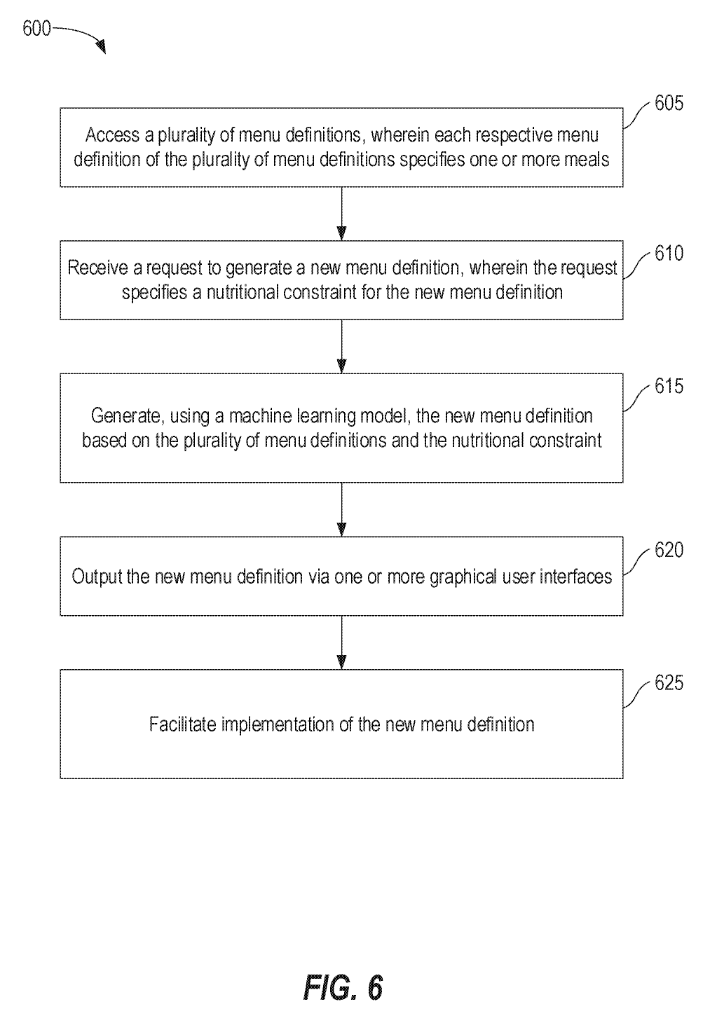

FIG. 6 is a flow diagram depicting an example method 600 for nutrition planning, according to some embodiments of the present disclosure. In some embodiments, the method 600 is performed by a machine learning system, such as the machine learning system 105 of FIG. 1 and/or the machine learning system 205 of FIG. 2.

At block 605, a plurality of menu definitions (e.g., the menu definitions 120 of FIG. 1) is accessed, wherein each respective menu definition of the plurality of menu definitions specifies one or more meals (e.g., the meals 310 of FIG. 3).

At block 610, a request to generate a new menu definition (e.g., the menu request 110 of FIG. 1) is received, wherein the request specifies a nutritional constraint (e.g., from the nutritional constraints 125 of FIG. 1) for the new menu definition.

At block 615, using a machine learning model (e.g., the machine learning model 215 of FIG. 2), the new menu definition (e.g., the new menu definition 115 of FIG. 1) is generated based on the plurality of menu definitions and the nutritional constraint.

At block 620, the new menu definition is output via one or more graphical user interfaces.

At block 625, implementation of the new menu definition is facilitated.

### Example Processing System for Nutrition Planning Machine Learning

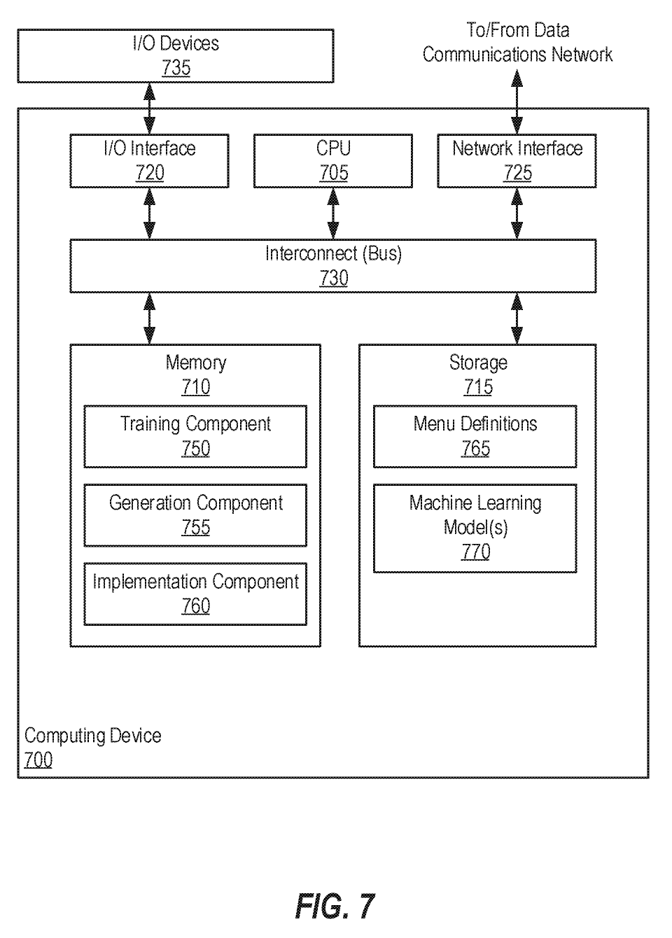

FIG. 7 depicts an example computing device 700 configured to perform various aspects of the present disclosure, according to some embodiments disclosed herein. Although depicted as a physical device, in embodiments, the computing device 700 may be implemented using virtual device(s), and/or across a number of devices (e.g., in a cloud environment). In one embodiment, the computing device 700 corresponds to a machine learning system, such as the machine learning system 105 of FIG. 1, the machine learning system 205 of FIG. 2, and/or the machine learning systems discussed above with reference to FIGS. 3-6.

As illustrated, the computing device 700 includes a CPU 705, memory 710, storage 715, a network interface 725, and one or more input/output (I/O) interfaces 720. In the illustrated embodiment, the CPU 705 retrieves and executes programming instructions stored in memory 710, as well as stores and retrieves application data residing in storage 715. The CPU 705 is generally representative of a single CPU and/or GPU, multiple CPUs and/or GPUs, a single CPU and/or GPU having multiple processing cores, and the like. The memory 710 is generally included to be representative of a random access memory. Storage 715 may be any combination of disk drives, flash-based storage devices, and the like, and may include fixed and/or removable storage devices, such as fixed disk drives, removable memory cards, caches, optical storage, network attached storage (NAS), or storage area networks (SAN).

In some embodiments, I/O devices 735 (such as keyboards, monitors, etc.) are connected via the I/O interface(s) 720. Further, via the network interface 725, the computing device 700 can be communicatively coupled with one or more other devices and components (e.g., via a network, which may include the Internet, local network(s), and the like). As illustrated, the CPU 705, memory 710, storage 715, network interface(s) 725, and I/O interface(s) 720 are communicatively coupled by one or more buses 730.

In the illustrated embodiment, the memory 710 includes a training component 750, a generation component 755, and an implementation component 760, which may perform one or more embodiments discussed above. Although depicted as discrete components for conceptual clarity, in embodiments, the operations of the depicted components (and others not illustrated) may be combined or distributed across any number of components. Further, although depicted as software residing in memory 710, in embodiments, the operations of the depicted components (and others not illustrated) may be implemented using hardware, software, or a combination of hardware and software.

In some embodiments, the training component 750 can be used to generate, train, and/or update machine learning models (e.g., the machine learning model 215 of FIG. 2), as discussed above. For example, the training component 750 may use training data (e.g., training exemplars generated based on historical menu definitions 220 of FIG. 2) to update or generate trained machine learning models, such that the models learn to generate new menu definitions, as discussed above.

In some embodiments, the generation component 755 can be used to generate new menu definitions (e.g., the new menu definition 115 of FIG. 1), as discussed above. For example, the generation component 755 may use trained machine learning models (e.g., the machine learning model 215 of FIG. 2) to generate new menu definitions.

In some embodiments, the implementation component 760 can be used to implement generated menu definitions, as discussed above. For example, the implementation component 760 may implement a nutrition plan by ordering or requesting the indicated quantities of various food items, scheduling delivery of the indicated food items on the corresponding days and/or at the corresponding times, and the like.

In the illustrated example, the storage 715 includes menu definitions 765 (which may correspond to the menu definitions 120 of FIG. 1 and/or the menu definitions 220 of FIG. 2). The storage 715 also includes machine learning model(s) 770 (which may correspond to the machine learning model 215 of FIG. 2). Although depicted as residing in storage 715, the depicted data may be stored in any suitable location, including memory 710.

Generally, the depicted components (and others not depicted) in memory 710 may evaluate and/or use the depicted data (and others not depicted) in storage 715 to provide accurate and efficient resupply attrition predictions, as discussed above.

### Example Clauses

Clause 1: A method, comprising: accessing a plurality of menu definitions, wherein each respective menu definition of the plurality of menu definitions specifies one or more meals; receiving a request to generate a new menu definition, wherein the request specifies a nutritional constraint for the new menu definition; generating, using a machine learning model, the new menu definition based on the plurality of menu definitions and the nutritional constraint; outputting the new menu definition via one or more graphical user interfaces; and facilitating implementation of the new menu definition.

Clause 2: A method according to Clause 1, wherein the new menu definition specifies a set of meals, from the plurality of menu definitions, that satisfy the nutritional constraint.

Clause 3: A method according to Clause 2, wherein generating the new menu definition comprises: selecting the set of meals; and evaluating the selected set of meals to confirm that the new menu definition satisfies the nutritional constraint.

Clause 4: A method according to any of Clauses 2-3, wherein each respective meal of the set of meals comprises a respective set of selections for a respective set of meal components.

Clause 5: A method according to any of Clauses 1-4, wherein: the nutritional constraint comprises a therapeutic dietary restriction for the new menu definition, and the therapeutic dietary restriction corresponds to users having at least one of: (i) diabetes, (ii) renal disease, or (iii) cardiovascular disease.

Clause 6: A method according to any of Clauses 1-5, wherein the request further specifies a number of meals to be included in the new menu definition.

Clause 7: A method according to any of Clauses 1-6, wherein the request further specifies a preferred expense of the new menu definition.

Clause 8: A method according to any of Clauses 1-7, wherein facilitating implementation of the new menu definition comprises receiving, from a user, one or more modifications to the new menu definition.

Clause 9: A method according to any of Clauses 1-8, wherein facilitating implementation of the new menu definition comprises ordering one or more food items based on the new menu definition.

Clause 10: Any patentable subject matter described herein.

Clause 11: A processing system, comprising: one or more processors; and one or more memories collectively comprising computer-executable instructions which, when executed on any combination of the one or more processors, cause the processing system to perform a method in accordance with any one of Clauses 1-10.

Clause 12: A system, comprising means for performing a method in accordance with any one of Clauses 1-10.

Clause 13: One or more non-transitory computer readable media collectively containing, in any combination, computer program code that, when executed by operation of a computing system, causes the computing system to perform a method in accordance with any one of Clauses 1-10.

Clause 14: A computer program product embodied on a computer-readable storage medium comprising code for performing a method in accordance with any one of Clauses 1-10.

### Additional Considerations

One or more elements or aspects or steps, or any portion(s) thereof, from one or more of any of claims below can be combined with one or more elements or aspects or steps, or any portion(s) thereof, from one or more of any of the other claims below or combinations thereof, to form one or more additional implementations and/or claims of the present disclosure.

While the present disclosure has been described with reference to one or more particular embodiments or implementations, those skilled in the art will recognize that many changes may be made thereto without departing from the spirit and scope of the present disclosure. Each of these implementations and obvious variations thereof is contemplated as falling within the spirit and scope of the present disclosure. It is also contemplated that additional implementations according to aspects of the present disclosure may combine any number of features from any of the implementations described herein.

The preceding description is provided to enable any person skilled in the art to practice the various embodiments described herein. The examples discussed herein are not limiting of the scope, applicability, or embodiments set forth in the claims. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. For instance, the methods described may be performed in an order different from that described, and various steps may be added, omitted, or combined. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, the word “exemplary” means “serving as an example, instance, or illustration.” Any aspect described herein as “exemplary” is not necessarily to be construed as preferred or advantageous over other aspects.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a c c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

Embodiments of the invention may be provided to end users through a cloud computing infrastructure. Cloud computing generally refers to the provision of scalable computing resources as a service over a network. More formally, cloud computing may be defined as a computing capability that provides an abstraction between the computing resource and its underlying technical architecture (e.g., servers, storage, networks), enabling convenient, on-demand network access to a shared pool of configurable computing resources that can be rapidly provisioned and released with minimal management effort or service provider interaction. Thus, cloud computing allows a user to access virtual computing resources (e.g., storage, data, applications, and even complete virtualized computing systems) in “the cloud,” without regard for the underlying physical systems (or locations of those systems) used to provide the computing resources.

Typically, cloud computing resources are provided to a user on a pay-per-use basis, where users are charged only for the computing resources actually used (e.g., an amount of storage space consumed by a user or a number of virtualized systems instantiated by the user). A user can access any of the resources that reside in the cloud at any time, and from anywhere across the Internet. In context of the present invention, a user may access applications or systems (e.g., the machine learning system) or related data available in the cloud. For example, the machine learning system could execute on a computing system in the cloud and train and/or use machine learning models to generate nutrition plans. In such a case, the machine learning system could receive and process the menu definitions, and store the models and definitions a storage location in the cloud. Doing so allows a user to access this information from any computing system attached to a network connected to the cloud (e.g., the Internet).

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. §112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. A method, comprising:
accessing a plurality of menu definitions, wherein each respective menu definition of the plurality of menu definitions specifies one or more meals;
receiving a request to generate a new menu definition, wherein the request specifies a nutritional constraint for the new menu definition;
generating, using a machine learning model, the new menu definition based on the plurality of menu definitions and the nutritional constraint;
outputting the new menu definition via one or more graphical user interfaces; and
facilitating implementation of the new menu definition.

2. The method of claim 1, wherein the new menu definition specifies a set of meals, from the plurality of menu definitions, that satisfy the nutritional constraint.

3. The method of claim 2, wherein generating the new menu definition comprises:
selecting the set of meals; and
evaluating the selected set of meals to confirm that the new menu definition satisfies the nutritional constraint.

4. The method of claim 2, wherein each respective meal of the set of meals comprises a respective set of selections for a respective set of meal components.

5. The method of claim 1, wherein:
the nutritional constraint comprises a therapeutic dietary restriction for the new menu definition, and
the therapeutic dietary restriction corresponds to users having at least one of: (i) diabetes, (ii) renal disease, or (iii) cardiovascular disease.

6. The method of claim 1, wherein the request further specifies a number of meals to be included in the new menu definition.

7. The method of claim 1, wherein the request further specifies a preferred expense of the new menu definition.

8. The method of claim 1, wherein facilitating implementation of the new menu definition comprises receiving, from a user, one or more modifications to the new menu definition.

9. The method of claim 1, wherein facilitating implementation of the new menu definition comprises ordering one or more food items based on the new menu definition.

10. A processing system, comprising:
one or more processors; and
one or more memories collectively comprising computer-executable instructions which, when executed on any combination of the one or more processors, cause the processing system to perform an operation comprising:
accessing a plurality of menu definitions, wherein each respective menu definition of the plurality of menu definitions specifies one or more meals;
receiving a request to generate a new menu definition, wherein the request specifies a nutritional constraint for the new menu definition;
generating, using a machine learning model, the new menu definition based on the plurality of menu definitions and the nutritional constraint;
outputting the new menu definition via one or more graphical user interfaces; and
facilitating implementation of the new menu definition.

11. The processing system of claim 10, wherein the new menu definition specifies a set of meals, from the plurality of menu definitions, that satisfy the nutritional constraint.

12. The processing system of claim 11, wherein generating the new menu definition comprises:
selecting the set of meals; and
evaluating the selected set of meals to confirm that the new menu definition satisfies the nutritional constraint.

13. The processing system of claim 11, wherein each respective meal of the set of meals comprises a respective set of selections for a respective set of meal components.

14. The processing system of claim 10, wherein:
the nutritional constraint comprises a therapeutic dietary restriction for the new menu definition, and
the therapeutic dietary restriction corresponds to users having at least one of: (i) diabetes, (ii) renal disease, or (iii) cardiovascular disease.

15. The processing system of claim 10, wherein facilitating implementation of the new menu definition comprises receiving, from a user, one or more modifications to the new menu definition.

16. The processing system of claim 10, wherein facilitating implementation of the new menu definition comprises ordering one or more food items based on the new menu definition.

17. One or more non-transitory computer readable media collectively containing, in any combination, computer program code that, when executed by operation of a computing system, performs an operation comprising:
accessing a plurality of menu definitions, wherein each respective menu definition of the plurality of menu definitions specifies one or more meals;
receiving a request to generate a new menu definition, wherein the request specifies a nutritional constraint for the new menu definition;
generating, using a machine learning model, the new menu definition based on the plurality of menu definitions and the nutritional constraint;
outputting the new menu definition via one or more graphical user interfaces; and
facilitating implementation of the new menu definition.

18. The one or more non-transitory computer readable media of claim 17, wherein:
the new menu definition specifies a set of meals, from the plurality of menu definitions, that satisfy the nutritional constraint, and
generating the new menu definition comprises:
selecting the set of meals; and
evaluating the selected set of meals to confirm that the new menu definition satisfies the nutritional constraint.

19. The one or more non-transitory computer readable media of claim 17, wherein facilitating implementation of the new menu definition comprises receiving, from a user, one or more modifications to the new menu definition.

20. The one or more non-transitory computer readable media of claim 17, wherein facilitating implementation of the new menu definition comprises ordering one or more food items based on the new menu definition.
